SEQUENCE LISTING (2 sequences)

SEQ ID NO: 1 - DNA, 3590 bp, Artificial Sequence
  Suppression cassette comprising the GZ-W64A promoter linked to the RGP silencing element, linked to the OLE promoter.
  promoter (1)...(1483): GZ-W64a promoter
  promoter (2635)...(3590): Oleosin promoter
  misc_feature (1491)...(2634): RGP1 silencing element
cgtatcacctatctaaataagtcacgggagtttcgaacgtccacttcgtcgcacggaattgcatgtttcttgttggaagcatattcacgcaatctccacacataaaggtttatgtataaacttacatttagctcagtttaattacagtcttatttggatgcatatgtatggttctcaatccatataagttagagtaaaaaataagtttaaattttatcttaattcactccaacatatatggatctacaatactcatgtgcatccaaacaaactacttatattgaggtgaatttggtagaaattaaactaacttacacactaagccaatctttactatattaaagcaccagtttcaacgatcgtcccgcgtcaatattattaaaaaactcctacatttctttataatcaacccgcactcttataatctcttctctactactataataagagagtttatgtacaaaataaggtgaaattatctataagtgttctggatattggttgttggctcccatattcacacaacctaatcaatagaaaacatatgttttattaaaacaaaatttatcatatatcatatatatatatatatcatatatatatataaaccgtagcaatgcacgggcatataactagtgcaacttaatacatgtgtgtattaagatgaataagagggtatccaaataaaaaacttgttgcttacgtatggatcgaaaggggttggaaacgattaaacgattaaatctcttcctagtcaaaattgaatagaaggagatttaatatatcccaatccccttcgatcatccaggtgcaaccgtataagtcctaaagtggtgaggaacacgaaagaaccatgcattggcatgtaaagctccaagaatttgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtatcggtggcataagaacacaagaaattgtgttaattaatcaaagctataaataacgctcgcatgcctgtgcacttctccatcaccaccactgggtcttcagaccattagctttatctactccagagcgcagaagaacccgatcgacagatatcggatccatggagatccatggcgggcacggtgacggtcccggggtcgtcgaccccctccacgccgctgctcaaggacgagctcgacatcgtgatcccgacgatccgcaacctcgacttcctggagatgtggcggcccttcttccagccctaccacctcatcatcgtgcaggacggcgacccgaccaagaccatcaaggtgcccgagggcttcgactacgaactctacaaccgcaacgacatcaaccgcatcctcgggcccatcgatatccgcgggcatgcctgcaggtcgactctagaacgaaggggttgctagccttgctgtgccagatgtatggcaggccagtcttgactcccaggctcaggtggtcgcagatgaccttcacacaccatcctgcccacatgtcgtcgtagcgaccgatgggctggccatcacccatgagaccaaagtacatagcagggccaatgagatccctgtcgaaggcaaggttcatgccacacatggggaacaaggttcccttggggattgtcatgacagcatcaacatacctctcattcctctccttgggcttgaccagctgtgtgggagcatcatagtcagggatgttcagccacaggccgtgggagacggcggtgtgagcaccctccctgaggctgaaggggtatccacgcacaaagtcagcaccctcacggtaggggtcgtacagggtgttgaagaagaacggggtggatgggctgaggaggttcttgatgtgctgctcaagagcattgatatccttgccagatgggtccttggcaacgaagcagtcgtcgtcgatggtgtagatgtacttcttcttggagaccatgtagccgaagcagcggcaggcggagtccttgaaggagatgcaggaggccttgggcccgaggatgcggttgatgtcgttgcggttgtagagttcgtagtcgaagccctcgggcaccttgatggtcttggtcgggtcgccgtcctgcacgatgatgaggtggtagggctggaagaagggccgccacatctccaggaagtcgaggttgcggatcgtcgggatcacgatgtcgagctcgtccttgagcagcggcgtggagggggtcgacgaccccgggaccgtcaccgtgcccgccatggatctgttaacccatggtagcgctagcagagcgagctaggtaccccacgtgcgcacgctgcccagagctcctgccgctgccgctgccgctgatgcttgagctacgactacgagtgaggtagatgagggcgagggagagctgggtgtttatagagggcgtggcaacgcgcggaggcgggatctggcgcggccgcgtgggacgcatgcgggactcgtgtcgggggcagcgcgacacctgtgtagggggtgtcgaggacaaggcttcctcggtcgcgccgcttccgcgcggcgatatatacgaggttgtgtgtgtgcgcgcacttccgttggatcggccgctggttgccggaggtggcggggccgaatctcatgtgggccgtagtccgggcctcttttacttcttttgtcttccgtgtctcactattttctggtccacgtagacctacatcacatactagcaagaactagtaaaagcatctgagcgtcagtatagttttagtatattaataataatatagatttattgtgactaaaatataatttgtggaaacaatgtgcttggtacctgttgagattgggtgaagaactacagcatgacaacatatttaaattgaggacacttcctctcttctccgaagaggatgaatggtagggtgtccagaaaacaaatttgtatatcgaaactcatgagaagttacgaagcgtaaaatatggattatcgtaattatttgctgaaggtgagatgcatgttctctcaatcgattaaccgtgtaatgctcattgccaataataatatcctagggaaagactcgatatatgatgaaagacaagacaacaataatcttcgccatcttataccttgggaggttctctaaacagggggtacacgggcgtagggatacaggaatgccaatcccgatagcagatatatttgaggtgtttgggtttggaggaatgagatagtcaatcggatc

SEQ ID NO: 2 - DNA, 1143 bp, Artificial Sequence
  RGP1 silencing element
ccatggcgggcacggtgacggtcccggggtcgtcgaccccctccacgccgctgctcaaggacgagctcgacatcgtgatcccgacgatccgcaacctcgacttcctggagatgtggcggcccttcttccagccctaccacctcatcatcgtgcaggacggcgacccgaccaagaccatcaaggtgcccgagggcttcgactacgaactctacaaccgcaacgacatcaaccgcatcctcgggcccatcgatatccgcgggcatgcctgcaggtcgactctagaacgaaggggttgctagccttgctgtgccagatgtatggcaggccagtcttgactcccaggctcaggtggtcgcagatgaccttcacacaccatcctgcccacatgtcgtcgtagcgaccgatgggctggccatcacccatgagaccaaagtacatagcagggccaatgagatccctgtcgaaggcaaggttcatgccacacatggggaacaaggttcccttggggattgtcatgacagcatcaacatacctctcattcctctccttgggcttgaccagctgtgtgggagcatcatagtcagggatgttcagccacaggccgtgggagacggcggtgtgagcaccctccctgaggctgaaggggtatccacgcacaaagtcagcaccctcacggtaggggtcgtacagggtgttgaagaagaacggggtggatgggctgaggaggttcttgatgtgctgctcaagagcattgatatccttgccagatgggtccttggcaacgaagcagtcgtcgtcgatggtgtagatgtacttcttcttggagaccatgtagccgaagcagcggcaggcggagtccttgaaggagatgcaggaggccttgggcccgaggatgcggttgatgtcgttgcggttgtagagttcgtagtcgaagccctcgggcaccttgatggtcttggtcgggtcgccgtcctgcacgatgatgaggtggtagggctggaagaagggccgccacatctccaggaagtcgaggttgcggatcgtcgggatcacgatgtcgagctcgtccttgagcagcggcgtggagggggtcgacgaccccgggaccgtcaccgtgcccgccatggatctgttaacccatgg

(19) United States
(12) Patent Application Publication
(10) Pub. No.: US 2007/0089200 A1
(43) Pub. Date: Apr. 19, 2007

(54) Manipulating the expression of reversibly glycosylated polypeptide (RGP) in plants

(75) Inventors: Xiaoming Bao, Johnston, IA (US); Darren B. Gruis, Baxter, IA (US); Craig Hastings, Perry, IA (US); George W. Singletary, Ankeny, IA (US); Deborah J. Wetterberg, Des Moines, IA (US)

Correspondence Address: ALSTON & BIRD LLP;PIONEER HI-BRED INTERNATIONAL, INC., BANK OF AMERICA PLAZA, 101 SOUTH TRYON STREET, SUITE 4000, CHARLOTTE, NC, 28280-4000, US

(73) Assignee: Pioneer Hi-Bred International, Inc., Johnston, IA (US)

(21) Appl. No.: 11/512,956

(22) Filed: Aug. 29, 2006

Related U.S. Application Data
(60) Provisional application No. 60/712,502, filed on Aug. 30, 2005.

(51) Int. Cl. A01H 5/00; C12N 15/82; C12N 5/04; A01H 1/00

(52) U.S. Cl. 800/284; 800/320.1; 435/468; 435/419

(57) ABSTRACT

Compositions and methods for altering the levels of reversibly glycosylated polypeptide (RGP) in plants are provided. The present invention recognizes that by altering the level of expression of RGP, plants and seeds having beneficial qualities can be obtained. The invention involves controlled suppression of RGP expression to obtain plants and seeds having decreased amounts of hemicellulose and arabinose. Controlled suppression of RGP, particularly in vegetative tissues and seeds, leads to plants with desirable characteristics, including altered cell wall structure, reduced grain fiber content, increased seed protein, increased oil content, and altered arabinoxylan or xyloglucan levels. Compositions of the invention include constructs for the suppression of RGP and transformed plants and seeds having the altered phenotype of the invention.

### CROSS-REFERENCE TO RELATED APPLICATIONS

This application claims the benefit of U.S. Provisional Application No. 60/712,502, filed Aug. 30, 2005, which is hereby incorporated herein in its entirety by reference.

### FIELD OF THE INVENTION

The present invention relates to the genetic modification of plants for improved characteristics, particularly to reducing the levels of reversibly glycosylated polypeptide (RGP) in plants.

### BACKGROUND OF THE INVENTION

The polysaccharides hemicellulose and pectin are major constituents of the cell wall that surrounds the outside of the plant cell plasma membrane. The primary structure of the cell wall comprises layers of cellulose microfibrils that are extensively cross-linked by hemicellulose polysaccharide chains. The hemicellulose branches help bind the microfibrils to one another and to other matrix components, thereby contributing to the tensile strength of the plant cell wall. The combination of this cell wall strength and pressure contributes to the rigidity of plant structures.

Reversibly glycosylated polypeptides (RGPs) belong to a family of self-glycosylating proteins. RGPs are unique to plants and appear to be highly conserved at the protein level. RGP genes have been isolated from many monocot and dicot plant species, including maize, rice, cotton, wheat, pea, and Arabidopsis. While the precise function of these glycosyltransferases has not been elucidated, reports indicate that these proteins may play a role in polysaccharide biosynthesis and may function in cell wall synthesis and/or in synthesis of starch. Indirect evidence suggests that RGP may be involved in hemicellulose biosynthesis. Further, because of the reversibility of nucleotide sugars attached to RGP, it is believed that RGPs are involved in the process of delivering sugar residues to glycosyltransferases. Until the present invention, no one has precisely controlled the expression of RGP in plants and noted the beneficial results. Therefore, methods are needed to control the expression of RGP in plants.

### SUMMARY OF THE INVENTION

Compositions and methods for altering the levels of reversibly glycosylated polypeptide (RGP) in plants are provided. The present invention recognizes that by altering the level of expression of RGP, plants and seeds having beneficial qualities can be obtained. The invention involves suppressing the levels of expression of RGP to obtain plants and seeds having decreased amounts of hemicellulose and arabinose. For purposes of the invention, RGP expression is suppressed by at least 50% but less than 100% as compared with normal expression levels in a comparable wild-type plant. Suppression of RGP, particularly in vegetative tissues and seeds, leads to plants with altered cell wall structure, reduced grain fiber content, increased seed protein, increased oil content, increased percent recovery of starch with the wet-milling process, improved soluble fiber content of grain for human consumption, improved feed conversion ratio of grain for livestock feed, and altered arabinoxylan or xyloglucan levels in plants. Thus, compositions of the invention include plants and seeds with improved growth rate and stalk or stem quality.

Promoters may be selected to provide for temporal or tissue-preferred suppression of RGP in plants. Compositions of the invention include constructs for the suppression of RGP and transformed plants and seeds having the altered phenotype of the invention.

### DETAILED DESCRIPTION OF THE INVENTION

Plants and seeds having beneficial agronomic characteristics are provided. The plants and seeds of the invention are obtained by controlling the level of expression of reversibly glycosylated polypeptide (RGP). While previous work has focused generally on altering the expression of RGP, the present invention is the first report to recognize that by precise suppression of the levels of RGP improvements in cell wall composition can be obtained. It is recognized that expression of RGP may be controlled by designing nucleotide constructs that suppress expression of RGP to the desired levels of the invention or, alternatively, plants having the phenotype of the invention can be selected after transformation or mutation. Beneficial agronomic characteristics of plants of the invention include increased extractability for the wet-milling process, improved soluble fiber content of grain, reduced overall grain fiber content, improved feed conversion ratio of grain for livestock feed, improved grain handling quality, reduced hemicellulosic arabinose residues, increased grain oil content, increased protein content, increased grain yield, improved grain nutritional value, and the like.

“Plants having the phenotype of the invention” are plants exhibiting reduced hemicellulose content and arabinose content. Such plants exhibit a reduction in hemicellulose by at least about 25%, at least about 30%, at least about 40%, at least about 50% as compared to a comparable wild-type plant. Likewise, plants having the phenotype of the invention exhibit a reduced arabinose/xylose ratio as compared to a comparable wild-type plant. The ratio is reduced by at least about 40%, at least about 50%, at least about 55%, at least about 60%, at least about 70% in transgenic plants of the invention. Specifically, plants of the invention have reduced expression levels of RGP as compared to wild-type plants. Typically, the level of expression of RGP is reduced by at least about 50%, about 60%, about 65%, about 70%, about 75%, about 80%, about 85%, about 90%, about 91%, about 92%, about 93%, about 94%, about 95%, about 96%, about 97%, about 98%, about 99%, but less than 100%, in the plant, resulting in reduction of arabinose levels by about 40% to about 70%, for example, by about 40%, about 45%, about 50%, about 55%, about 60%, about 65%, or about 70%. In some embodiments, the level of expression of RGP is reduced by at least about 50%, about 60%, about 65%, up to about 70% in the plant. Methods to identify plants having the phenotype of the invention include western blots measuring levels of RGP protein as well as analysis of hemicellulose sugar content in the plant and seed.

The methods of the invention provide plants and seeds having reduced RGP expression. RGP has been indicated to be involved in hemicellulose biosynthesis. While not bound by any particular mechanism of action, the invention provides evidence that suppression of RGP results in reduction of hemicellulosic arabinose and hemicellulose having a direct impact on the physical properties of the cell wall. Further, protein and oil content is higher in transgenic kernels along with an increase in kernel weight. In this manner, RGPs provide another tool to manipulate the cell wall and increase yield.

Because suppressing RGP results in the reduction of arabinose, the structure and cross-linking pattern of the cell wall of the transgenic plants and seeds will be different from wild-type plants and seeds. Thus, the transgenic plants and seeds of the invention are beneficial in having a reduction in overall grain fiber content, increased grain protein content, increased grain oil content, increased grain yield (as indicated by increased kernel weight), improved grain nutritional value and available energy for animal feed, improved grain nutritional value for food, improved grain digestibility for animal feed, improved baking qualities, improved grain qualities for dry grind ethanol, improved grain qualities for beer production, and improved grain qualities for wet milling.

By “reduces” or “reducing” the level of a polynucleotide or a polypeptide encoded thereby is intended to mean, the polynucleotide level or polypeptide level of the target sequence (i.e., RGP) is statistically lower than the polynucleotide level or polypeptide level of the same target sequence in an appropriate control plant that is not expressing the nucleotide construct of the invention. In particular embodiments of the invention, reducing the polynucleotide level and/or the polypeptide level of the target sequence in a modified plant according to the invention results in less than about 50%, less than about 40%, but at least about 30% of the polynucleotide level, or the level of the polypeptide encoded thereby, of the same target sequence in an appropriate control or wild-type plant. In other embodiments, reducing the polynucleotide level and/or the polypeptide level of the target sequence in a modified plant according to the invention results in less than about 50%, less than about 40%, less than about 30%, less than about 20%, less that about 15%, less than about 10%, less than about 5%, but at least 1% of the polynucleotide level, or the level of the polypeptide encoded thereby, of the same target sequence in an appropriate control or wild-type plant. Methods to assay for the level of the RNA transcript, the level of the encoded polypeptide, or the activity of the polynucleotide or polypeptide are discussed elsewhere herein.

As shown herein, suppressing the expression of RGP in a seed-preferred manner decreases hemicellulose and arabinose biosynthesis. Nucleotide constructs that provide for expression of RNA transcripts that inhibit RGP (i.e., RGP inhibitory transcripts) are described herein and include suppression cassettes that are capable of expressing RNA transcripts that form stem-loop structures, for example, constructs comprising the sequence set forth in SEQ ID NO:2 and described herein below in Example 1. A discussion of the suppression cassette used in Example 1 is described in U.S. Application No. 60/712,354, entitled “Compositions and Methods for Modulating Expression of Gene Products,” filed Aug. 30, 2006, and copending U.S. Utility application Ser. No. ______, also entitled “Compositions and Methods for Modulating Expression of Gene Products,” filed concurrently herewith. This suppression cassette is advantageous as it provides an efficient means to inhibit RGP expression in spatially separated tissues within a seed, for example, within the endosperm (i.e., with a gamma-zein or Opaque-2 promoter) and the embryo (i.e., with a globulin 1, oleosin, or EAP1 promoter), and can provide for expression of the inhibitory RNA transcripts throughout early and late seed development. The results described herein below illustrate embodiments wherein suppression of RGP was targeted in both the embryo and endosperm of the transformed seed by using the oleosin promoter and the gamma-zein promoter. In other embodiments, suppression of RGP occurs during early seed development using, for example, the eep1 or eep2 promoter, and during late seed development using, for example, an oleosin promoter. Use of these nucleotide constructs in the methods of the present invention can provide for decreased hemicellulose and/or arabinose content in the seed or part thereof, for example, endosperm and embryo, and/or throughout early and late seed development.

RGPs are polypeptides unique to plants. They belong to a family of self-glycosylating proteins. Before the present invention, the precise function of these glycosyltransferases had not been elucidated. While RGPs had been reported from a number of plants and reports of manipulation of RGP are in the literature, no one had effectively controlled suppression of RGP expression and observed the beneficial results. Preferred compositions of the invention include plants, plant tissues, and plant seeds having RGP suppressed at high levels. That is, the level of RGP being expressed is only enough to allow for the germination and growth of the plant. Thus, plants of the invention have levels of expression of RGP reduced by about 50% and up to but less than 100%, including about 50% to about 99%, about 50% to about 95%, about 50% to about 90%, about 50% to about 85%, about 50% to about 80%, about 50% to about 75%, and about 50% to about 70%, resulting in reduction of arabinose levels by about 40% to about 70%. In some embodiments, plants of the invention have levels of expression of RGP reduced by about 50% to about 70%, about 60% to about 70%, about 65% to about 70%, including about 66%, about 67%, about 68%, about 69%, up to about 70%.

RGPs have been isolated from both dicots and monocots and are highly conserved at the protein level. GenBank EST accession numbers for RGP sequences include: Arabidopsis A042694, H37657, H76915, N37306, N65528, N65622, R30021, R90614, T04300, T20512, T22507, T22943, T23020, T42672, T44394, T44917, T46245, T46745, Z17897, Z37199, Z38054; rice D15688, D15900, D23283, D24192, D25050, D28284, D39893, D40029, D40099, D40369, D42010; corn W21688; (Glycine max) A1441631, BE210760, AW620577, BM17751 1, BF596821, BE608213, BE47392, BM094929, BM885700, BM732704; and (Glycine soja) BG044039, BF597555. See also, Dhugga et al. (1997) Proc. Natl. Acad. Sci USA 94(14):7679-7684; Zhao and Liu (2002) Biochima et Biophysica Acta 1574:370-374; Langeveld et al. (2002) Plant Physiol. 129(1):278-289; Delgado et al. (1998) Plant Physiol. 116(4): 1339-1350. Such sequences provided in the accession numbers and in the references are herein incorporated by reference.

Because of the homology of the sequences across plant species, a nucleotide construct comprising a polynucleotide that is designed to suppress expression of RGP from one species may function to suppress expression of RGP in a number of plants. Thus, the invention encompasses utilizing nucleotide constructs comprising polynucleotides that are homologous or heterologous to the plant in which the RGP is being suppressed. That is, the suppression polynucleotide used in corn may be derived from the corn RGP sequence (homologous) or alternatively, may be derived from another plant RGP sequence (heterologous). In other embodiments, a synthetic sequence may be utilized having homology to the plant RGP such that the sequence functions to suppress expression in one or a number of plant species. It is recognized that any of the RGP sequences in the art, or fragments and variants thereof, can be used to design nucleotide constructs that are capable of expressing a polynucleotide that suppresses expression of RGP.

Thus, fragments and variants of RGP polynucleotides are also encompassed by the present invention. By “fragment” is intended a portion of the polynucleotide. Fragments of a polynucleotide that are useful as hybridization probes or in the nucleotide constructs of the invention generally do not retain biological activity. Thus, fragments of a nucleotide sequence may range from at least about 20 nucleotides, about 50 nucleotides, about 100 nucleotides, and up to the full-length RGP polynucleotide. “Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polyiiucleotide or polypeptide comprises a naturally occurring nucleotide sequence or amino acid sequence, respectively. Variant polynucleotides also include synthetically derived polynucleotides. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al. (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see Henikoff and Henikoff (1989) Proc. Natl. Acad. Sci. USA 89:10915).

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As indicated, the present invention is based on the suppression of RGP in plants to obtain plants having an optimal RGP phenotype. By “suppression of RGP” in a plant means reducing the expression of RGP. It is recognized that any means of suppression of the gene can be utilized in the practice of the invention as discussed below. Transformed plants can be selected for those that have the appropriate phenotype. Methods to select the plant include measuring the RNA transcript levels of RGP in the plant, western blot analysis of the RGP protein levels in the plant, measurement of hemicellulose or arabinose levels in the plant, and the like.

In accordance with the methods of the invention, the expression level of the RGP protein is reduced by introducing into the plant a nucleotide construct that expresses a polynucleotide that inhibits the expression of RGP. The polynucleotide may inhibit the expression of RGP directly, by preventing translation of the RGP messenger RNA, or indirectly, by encoding a polypeptide that inhibits the transcription or translation of a plant gene encoding a RGP. Methods for inhibiting the expression of a gene in a plant are well known in the art, and any such method may be used in the present invention to inhibit the expression of RGP.

In accordance with the present invention, the expression of an RGP is inhibited if the protein level of the RGP is statistically lower than the protein level of the same RGP in a comparable wild-type plant that has not been genetically modified or mutagenized to inhibit the expression of that RGP. In the practice of the invention, the protein level of the RGP in a modified plant is optimally manipulated to precise levels. The resulting plant or plant seed exhibits reduced expression levels of the RGP but retains enough levels of the protein to germinate and grow. As previously indicated, the level of the RGP will be reduced by at least about 50% but less than 100%, including at least about 50% to about 99%, about 50% to about 95%, about 50% to about 90%, about 50% to about 85%, about 50% to about 80%, about 50% to about 75%, and about 50% to about 70%, resulting in reduction of arabinose levels by about 40% to about 70%. In some embodiments, plants of the invention have levels of expression of RGP reduced by about 50% to about 70%, including about 50%, about 55%, about 60%, about 61%, about 62%, about 63%, about 64%, about 65%, about 66%, about 67%, about 68%, about 69%, or up to about 70% relative to the level of expression of the same RGP in a plant that is not a mutant or that has not been genetically modified to inhibit the expression of that RGP. The expression level of the RGP may be measured directly, for example, by assaying for the level of RGP expressed in the plant cell or plant, or indirectly, for example, by measuring the RNA or protein levels of the RGP in the plant cell or plant.

Now that the present invention has recognized that there is a benefit to the plant and plant products when RGP expression is reduced to optimal levels (reduced by at least 50% but not greater than about 70% reduction), any means to suppress the expression of RGP may be utilized to produce plants of the invention. Non-limiting examples of methods of reducing the expression of RGP are discussed below.

A. Polynucleotide-Based Methods:

In some embodiments of the present invention, a plant cell is transformed with a nucleotide construct that is capable of expressing a polynucleotide that inhibits the expression of RGP. The term “expression” as used herein refers to the biosynthesis of a gene product, including the transcription and/or translation of said gene product. For example, for the purposes of the present invention, a nucleotide construct capable of expressing a polynucleotide that inhibits the expression of RGP is a construct capable of producing an RNA molecule that inhibits the transcription and/or translation of RGP. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of an RGP are given below.

1. Sense Suppression/Cosuppression

In some embodiments of the invention, inhibition of the expression of RGP may be obtained by sense suppression or cosuppression. For cosuppression, a nucleotide construct is designed to express an RNA molecule corresponding to all or part of a messenger RNA encoding an RGP in the “sense” orientation. Overexpression of the RNA molecule can result in reduced expression of the native gene. Accordingly, multiple plant lines transformed with the cosuppression construct are screened to identify those that show the optimal inhibition of RGP expression, i.e., wherein the level of expression of RGP is reduced by at least about 50% but less than 100%, including at least about 50% to about 99%, about 50% to about 95%, about 50% to about 90%, about 50% to about 85%, about 50% to about 80%, about 50% to about 75%, and about 50% to about 70% relative to a comparable control or wild-type plant, resulting in reduction of arabinose levels by about 40% to about 70% relative to a comparable control or wild-type plant.

The polynucleotide used for cosuppression may correspond to all or part of the sequence encoding the RGP, all or part of the 5′ and/or 3′ untranslated region of an RGP transcript, or all or part of both the coding sequence and the untranslated regions of a transcript encoding RGP. In some embodiments where the polynucleotide comprises all or part of the coding region for RGP, the nucleotide construct is designed to eliminate the start codon of the polynucleotide so that no protein product will be transcribed.

Cosuppression may be used to inhibit the expression of plant genes to produce plants having undetectable protein levels for the proteins encoded by these genes. See, for example, Broin et al. (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell et al. (1994) Proc. Natl. Acad. Sci. USA 91:3490-3496; Jorgensen et al. (1996) Plant Mol. Biol. 31:957-973; Johansen and Carrington (2001) Plant Physiol. 126:930-938; Broin et al. (2002) Plant Cell 14:1417-1432; Stoutjesdijk et al. (2002) Plant Physiol. 129:1723-1731; Yu et al. (2003) Phytochemistry 63:753-763; and U.S. Pat. Nos. 5,034,323, 5,283,184, and 5,942,657; each of which is herein incorporated by reference. The efficiency of cosuppression may be increased by including a poly-dT region in the nucleotide construct at a position 3′ to the sense sequence and 5′ of the polyadenylation signal. See, U.S. Patent Publication No. 20020048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323; herein incorporated by reference.

2. Antisense Suppression

In some embodiments of the invention, inhibition of the expression of the RGP may be obtained by antisense suppression. For antisense suppression, the nucleotide construct is designed to express an RNA molecule complementary to all or part of a messenger RNA encoding the RGP. Overexpression of the antisense RNA molecule can result in reduced expression of the native gene. Accordingly, multiple plant lines transformed with the antisense suppression construct are screened to identify those that show the greatest inhibition of RGP expression.

The polynucleotide for use in antisense suppression may correspond to all or part of the complement of the sequence encoding the RGP, all or part of the complement of the 5′ and/or 3′ untranslated region of the RGP transcript, or all or part of the complement of both the coding sequence and the untranslated regions of a transcript encoding the RGP. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550, or greater may be used. Methods for using antisense suppression to inhibit the expression of endogenous genes in plants are described, for example, in Liu et al. (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the nucleotide construct at a position 3′ to the antisense sequence and 5′ of the polyadenylation signal. See, U.S. Patent Publication No. 20020048814, herein incorporated by reference.

3. Double-Stranded RNA Interference

In some embodiments of the invention, inhibition of the expression of an RGP may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the nucleotide construct to comprise both a sense sequence and an antisense sequence. Alternatively, separate constructs may be used for the sense and antisense sequences. Multiple plant lines transformed with the dsRNA interference construct or constructs are then screened to identify plant lines that show the optimal inhibition of RGP expression. Methods for using dsRNA interference to inhibit the expression of endogenous plant genes are described in Waterhouse et al. (1998) Proc. Natl. Acad. Sci. USA 95:13959-13964, Liu et al. (2002) Plant Physiol. 129:1732-1743, and WO 99/49029, WO 99/53050, WO 99/61631, and WO 00/49035; each of which is herein incorporated by reference.

4. Hairpin RNA Interference and Intron-Containing Hairpin RNA Interference

In some embodiments of the invention, inhibition of the expression of RGP may be obtained by hairpin RNA (hpRNA) interference or intron-containing hairpin RNA (ihpRNA) interference. These methods are highly efficient at inhibiting the expression of endogenous genes. See, Waterhouse and Helliwell (2003) Nat. Rev. Genet. 4:29-38 and the references cited therein.

For hpRNA interference, the nucleotide construct of the invention is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz (2000) Proc. Natl. Acad Sci. USA 97:4985-4990; Stoutjesdijk et al. (2002) Plant Physiol. 129:1723-1731; and Waterhouse and Helliwell (2003) Nat. Rev. Genet. 4:29-38. Methods for using hpRNA interference to inhibit or silence the expression of genes are described, for example, in Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, and U.S. Patent Publication No. 20030175965; each of which is herein incorporated by reference. A transient assay for the efficiency of hpRNA constructs to silence gene expression in vivo has been described by Panstruga et al. (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith et al. (2000) Nature 407:319-320. In fact, Smith et al. show 100% suppression of endogenous gene expression using ihpRNA-mediated interference. Methods for using ihpRNA interference to inhibit the expression of endogenous plant genes are described, for example, in Smith et al. (2000) Nature 407:319-320; Wesley et al. (2001) Plant J. 27:581-590; Wang and Waterhouse (2001) Curr. Opin. Plant Biol. 5:146-150; Waterhouse and Helliwell (2003) Nat. Rev. Genet. 4:29-38; Helliwell and Waterhouse (2003) Methods 30:289-295, and U.S. Patent Publication No. 20030180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 02/00904, herein incorporated by reference.

Transcriptional gene silencing (TGS) may be accomplished through use of hpRNA constructs wherein the inverted repeat of the hairpin shares sequence identity with the promoter region of a gene to be silenced. Processing of the hpRNA into short RNAs which can interact with the homologous promoter region may trigger degradation or methylation to result in silencing (Aufsatz et al. (2002) PNAS 99 (Suppl. 4): 16499-16506; Mette et al. (2000) EMBO J 19(19):5194-5201).

5. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for RGP). Methods of using amplicons to inhibit the expression of endogenous plant genes are described, for example, in Angell and Baulcombe (1997) EMBO J 16:3675-3684, Angell and Baulcombe (1999) Plant J. 20:357-362, and U.S. Pat. No. 6,646,805, each of which is herein incorporated by reference.

6. Ribozymes

In some embodiments, the polynucleotide expressed by the nucleotide construct of the invention is catalytic RNA or has ribozyme activity specific for the messenger RNA of RGP. Thus, the polynucleotide causes the degradation of the endogenous messenger RNA, resulting in reduced expression of RGP. This method is described, for example, in U.S. Pat. No. 4,987,071, herein incorporated by reference.

7. Small Interfering RNA or Micro RNA

In some embodiments of the invention, inhibition of the expression of RGP may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example Javier et al. (2003) Nature 425: 257-263, herein incorporated by reference.

For miRNA interference, the nucleotide construct is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of RGP expression, the 22-nucleotide sequence is selected from a RGP transcript sequence and contains 22 nucleotides of the RGP sequence in sense orientation and 21 nucleotides of a corresponding antisense sequence that is complementary to the sense sequence. miRNA molecules are highly efficient at inhibiting the expression of endogenous genes, and the RNA interference they induce is inherited by subsequent generations of plants.

B. Polypeptide-Based Inhibition of Gene Expression

In one embodiment, the nucleotide construct of the invention comprises an expression cassette that comprises a polynucleotide that encodes a zinc finger protein that binds to a gene encoding an RGP, resulting in reduced expression of the gene. In particular embodiments, the zinc finger protein binds to a regulatory region of an RGP gene. In other embodiments, the zinc finger protein binds to a messenger RNA encoding an RGP and prevents its translation. Methods of selecting sites for targeting by zinc finger proteins have been described, for example, in U.S. Pat. No. 6,453,242, and methods for using zinc finger proteins to inhibit the expression of genes in plants are described, for example, in U.S. Patent Publication No. 20030037355; each of which is herein incorporated by reference.

C. Polyepeptide-Based Inhibition of Protein Activity:

In some embodiments of the invention, the nucleotide construct of the invention comprises an expression cassette comprising a polynucleotide that encodes an antibody that binds to RGP, and reduces the activity of the RGP. In another embodiment, the binding of the antibody results in increased turnover of the antibody-RGP complex by cellular quality control mechanisms. The expression of antibodies in plant cells and the inhibition of molecular pathways by expression and binding of antibodies to proteins in plant cells are well known in the art. See, for example, Conrad and Sonnewald (2003) Nature Biotech. 21:35-36, incorporated herein by reference.

Thus any method known in the art for reducing expression level of a polypeptide of interest can be used to practice the methods of the present invention.

Nucleotide constructs for use in the methods of the invention include expression cassettes for expression of transcripts that suppress RGP expression in the plant of interest. The cassette will include 5′ and 3′ regulatory sequences operably linked to a polynucleotide capable of inhibiting or suppressing expression of RGP. “Operably linked” is intended to mean a functional linkage between two or more elements. For example, an operable linkage between a polynucleotide of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the polynucleotide of interest to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a polynucleotide capable of suppression of RGP, and a transcriptional and translational termination region (i.e., termination region) functional in plants. The regulatory regions (i.e., promoters, transcriptional regulatory regions, and translational termination regions) and/or the polynucleotide capable of suppression may be native/analogous to the host cell or to each other. Alternatively, the regulatory regions and/or the polynucleotide capable of suppression may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

The termination region may be native with the transcriptional initiation region, may be native with the plant host, or may be derived from another source (i.e., foreign or heterologous to the promoter, the plant host, or any combination thereof). Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention, including the native promoter of the polynucleotide sequence of interest. The promoters can be selected based on the desired outcome. That is, promoters will be selected based on whether tissue-preferred suppression, temporal suppression, or constitutive suppression of RGP is desired.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to suppress RGP within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 1 12(3):1331-1341; Van Camp et al. (1996) Plant Physiol. 1 12(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108, herein incorporated by reference. Such seed-preferred promoters include, but are not limited to, Cim 1 (cytokinin-induced message); cZ19B1 (maize 19 kDa zein); milps (myo-inositol-1-phosphate synthase) (see WO 00/11177 and U.S. Pat. No. 6,225,529; herein incorporated by reference). Gamma-zein is an endosperm-specific promoter. Globulin 1 (Glb-1) is a representative embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, Globulin 1, etc. See also WO 00/12733, where seed-preferred promoters from end1 and end2 genes are disclosed; herein incorporated by reference.

Where low level expression is desired, weak promoters will be used. Generally, by “weak promoter” is intended a promoter that drives expression of a coding sequence at a low level. By low level is intended at levels of about 1/1000 transcripts to about 1/100,000 transcripts to about 1/500,000 transcripts. Alternatively, it is recognized that weak promoters also encompasses promoters that are expressed in only a few cells and not in others to give a total low level of expression. Where a promoter is expressed at unacceptably high levels, portions of the promoter sequence can be deleted or modified to decrease expression levels.

Such weak constitutive promoters include, for example, the core promoter of the Rsyn7 promoter (WO 99/43838 and U.S. Pat. No. 6,072,050), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463; and 5,608,142. See also, U.S. Pat. No. 6,177,611, herein incorporated by reference.

In some embodiments of the invention, the methods involve introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration (see, for example, U.S. Pat. Nos. 4,945,050; U.S. Pat. No. 5,879,918; U.S. Pat. No. 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lecd transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide of the invention can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The present invention may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide of the invention, for example, an expression cassette of the invention, stably incorporated into their genome.

The plants and seeds of the invention have improved digestibility. “Digestibility” is defined herein as the fraction of the feed or food that is not excreted as feces. It can be further defined as digestibility of specific components (such as energy or protein) by determining the concentration of these components in the foodstuff and in the excreta. Digestibility can be estimated using in vitro assays, which are routinely done to screen large numbers of different food ingredients and plant varieties. In vitro techniques, including assays with rumen inocula and/or enzymes for ruminant livestock (e.g., Tilley and Terry, 1963; Pell and Schofield) and various combinations of enzymes for monogastric animals reviewed in Boisen and Eggus (1991) are also useful techniques for screening transgenic materials for which only limited sample is available.

In specific embodiments, the plants of the invention find use in the wet milling industry. In the wet milling process, the purpose is to fractionate the kernel and isolate chemical constituents of economic value into their component parts. The process allows for the fractionation of starch into a highly purified form, as well as, for the isolation in crude forms of other material including, for example, unrefined oil, or as a wide mix of materials which commonly receive little to no additional processing beyond drying. Hence, in the wet milling process grain is softened by steeping and cracked by grinding to release the germ from the kernels. The germ is separated from the heavier density mixture of starch, hulls and fiber by “floating” the germ segments free of the other substances in a centrifugation process. This allows a clean separation of the oil-bearing fraction of the grain from tissue fragments that contain the bulk of the starch. Since it is not economical to extract oil on a small scale, many wet milling plants ship their germ to large, centralized oil production facilities. Oil is expelled or extracted with solvents from dried germs and the remaining germ meal is commonly mixed into corn gluten feed (CGF), a coproduct of wet milling. Hence, starch contained within the germ is not recovered as such in the wet milling process and is channeled to CGF. See, for example, Anderson et al. (1982) “The Corn Milling Industry”; CRC Handbook ofProcessing and Utilization in Agriculture, A. Wolff, Boca Raton, Fla., CRC Press., Inc., Vol. 11, Part 1, Plant Products: 31-61 and Eckhoff (June 24-26, 1992) Proceedings of the 4th Corn Utilization Conference, St. Louis, Mo., printed by the National Corn Growers Association, CIBA-GEIGY Seed Division, and the USDA, both of which are herein incorporated by reference.

The following examples are offered by way of illustration and not by way of limitation.

### Experimental

### EXAMPLE 1

### Suppression Cassette Provides for Differential Promoter Expression of Reversibly Glycosylated Polyepttide-1 (RGP 1)

The maize RGPI gene encodes a polypeptide that is involved in hemicellulose production (see, for example, U.S. Pat. No. 6,194,638, herein incorporated by reference in its entirety). A nucleotide construct comprising a suppression cassette targeting RGP1 expression in both endosperm and embryo was designed. In this manner, an inverted repeat comprising a sense and antisense sequence of the maize RGP1 gene was created using standard molecular biology protocols. A 277 base pair (bp) HindIII-ApaI fragment from the 5′ end of the RGP1 coding sequence was ligated into a cloning intermediate. This plasmid was restriction digested, the end filled in with Klenow enzyme, and then digested with a second restriction enzyme. Into this backbone, a second fragment of RGP1 (an 848 bp BamHI/HpaI fragment, also from the 5′ end of the coding sequence) was ligated, such that the second fragment was in reverse orientation relative to the first fragment. The suppression cassette was created by moving the promoter from the maize 16 kDa oleosin gene (OLE PRO; a BamHI/BglII fragment (969 bp)) into a cloning intermediate vector. From this, the OLE PRO was moved as a 997 bp HpaI/HindIII fragment into a GZ-W64A PRO cassette (promoter from the maize 27 kDa gamma-zein gene), replacing the GZ-W64A terminator sequence. The resulting vector comprised the two promoters directed toward each other, separated by a multi-cloning site. The RGP1 inverted repeat fragment was ligated as a 1129 bp BamHI fragment into BglII-digested plasmid. The entire promoter:inverted repeat:promoter suppression cassette (SEQ ID NO: 1) was finally moved as a BstEII fragment into a BstEII-digested binary vector. This plasmid was transferred by electroporation into electro-competent Agrobacterium tumefaciens cells, where cos-specific recombination with a resident vir plasmid resulted in the formation of a cointegrate plasmid. Immature embryos of maize (GS3×HG69) were transformed using Agrobacterium tumefaciens cells carrying Plasmid B.

Hemicellulose Assay

Seed Dissection

Nineteen mature kernels from each transformation event were soaked overnight in water at 4° C. The seeds were cut in half and dissected into embryo and endosperm. The dissected embryo and endosperm were dried in a lyophilizer. One-half of each endosperm or embryo was used for Western blotting and the remaining half was used for hemicellulose analysis.

Western Blots

One-half of the embryo or endosperm was placed into a 96-well matrix snap rack (Matrix Technologies #4147). The tissue was ground in the Spex Certiprep GenoGrinder for two minutes at 1400 strokes/minute or until ground. One milliliter of extraction buffer (50 mM Tris, 100 mM DTT, 2% SDS) was added to each endosperm sample or 0.5 milliliter for embryo samples. The samples were ground again for 1 minute at 1400 strokes/minute in the GenoGrinder. The samples were heated at 100° C. for 5 minutes and centrifuged at 4,000 rpm for 10 minutes. Thirty microliters of supernatant was added to 10 μl of 4× E-PAGE loading dye Buffer 1 (Invitrogen catalog #EPBUF-01). Ten microliters was loaded onto Invitrogen's E-PAGE 96-well gel (catalog #EP096-06), and the gel was run for 14 minutes. For the Western blot, the proteins were transferred to PVDF membrane using semi-dry blotting apparatus for 1.5 hours at 0.8 mA/cm². The primary antibody used for the Western blot was a 1:5000 dilution of (X-RGP1 antibody from pea. The secondary antibody was goat a-rabbit IgG (H+L)-HRP conjugated (BioRad #170-6515). The blot was developed using Amersham Biosciences ECL Western blotting detection reagents kit (RPN2106).

Based on the results of the Western blots, events were screened for transformants. The results of the first screen are shown in Table 1.

TABLE 1

| Event | Knockdown | WT:T Ratio |
|---|---|---|
| 1 | fair/weak | 4:4 |
| 2 | fair | 3:5 |
| 3 | fair | 2:6 |
| 4 | fair | 4:4 |
| 5 | strong | 2:6 |
| 6 | fair/weak | 4:4 |
| 7 | strong | 3:5 |
| 8 | fair/strong | 4:4 |
| 9 | strong | 5:3 |
| 10 | fair/strong | 4:4 |
| 11 | fair/strong | 4:4 |
| 12 | fair | 4:4 |
| 13 | fair | 4:4 |
| 14 | strong | 5:3 |
| 15 | strong | 5:3 |

Sample Preparation for Analysis of Hemicellulose Sugars

The remaining ½ embryo or ½ endosperm was pooled into wild-type or transgenic for each event based on the Western blot results. The pooled endosperm or embryo was ground in the Gendogrinder into a powder. Fifty milligram samples were weighed out for hemicellulose analysis. Soluble sugars were removed by adding 1 ml 80% ethanol and a small stir bar to each 50-mg sample of ground tissue. The samples were vortexed and heated at 100° C. for one minute. Samples were centrifuged at 14,000 rpm for 10 minutes and the supernatant was discarded. To the pellet, 1 ml of acetone was added, and the samples were vortexed and centrifuged at 14,000 rpm for 10 minutes. The supernatant was discarded and the pellets were dried. The pellets were de-starched by adding 0.3 ml α-amylase solution (300 units/assay a-amylase in 50 mM MOPS (pH 7.0), 5 mM calcium chloride, 0.02% sodium-azide) and heated at 90-95° C. for 10 minutes with constant stirring using a magnetic stir plate. Then 0.2 ml amyloglucosidase (Boehringer Manheim from Aspergillus niger catalog #1202367) solution (20 U/assay amyloglucosidase in 285 mM Sodium-acetate pH 4.5 0.02% Sodium-azide) was added to each tube and incubated at 55° C. overnight. Absolute ethanol was added to each tube to a final centration of 70%, the samples were vortexed and centrifuged at 14,000 rpm for 10 minutes. The pellet was washed two times with 1 ml 80% ethanol discarding the supernatant each time. The pellet was washed with 1 ml acetone and left to dry. To hydrolyze the hemicellulose sugars, 1 ml of 1 M sulfuric acid was added, and the samples were heated at 100° C. for 30 minutes. The samples were cooled on ice and spun at 14,000 mm for 10 minutes. The resulting supernatant was used for hemicellulose sugar analysis.

The average control weight of transformed kernel was tabulated (see Table 2) and the sugar content assayed.

TABLE 2

| | | Average WT | Average T | |
|---|---|---|---|---|
| | | Kernel wt. | Kernel wt. | % Control |
| Event | WT:T Ratio | (mg) | (mg) | Avg. wt. |
| 1 | 9:9 | 160.02 | 149.94 | 93.70 |
| 3 | 8:10 | 350.72 | 346.82 | 98.89 |
| 4 | 7:11 | 236.89 | 234.76 | 99.10 |
| 5 | 13:5 | 311.38 | 312.46 | 100.35 |
| 7 | 9:9 | 284.33 | 244.79 | 86.09 |
| 8 | 5:13 | 293.37 | 266.27 | 90.76 |
| 9 | 6:12 | 266.45 | 258.14 | 96.88 |
| 10 | 10:8 | 290.57 | 284.67 | 97.97 |
| 11 | 7:11 | 295.63 | 301.14 | 101.86 |
| 12 | 3:13 | 253.72 | 273.88 | 107.95 |
| 13 | 6:9 | 278.83 | 278.81 | 99.99 |
| 14 | 4:13 | 273.75 | 249.15 | 91.01 |
| 15 | 12:2 | 320.81 | 331.36 | 103.29 |
| Total | | 278.19 | 271.70 | 97.67 |

Analysis of High Performance Anion Exchange Chromatography with Pulsed Amperometric Detection (HPAEC PAD)

HPAEC was used for separation, identification, and quantitation of arabinose, galactose, glucose, xylose, and mannose. A Dionex DX500 high performance liquid chromatograph (HPLC) equipped with a GP40 or GP50 pump, ED40 electrochemical detector, pulsed amperometric detector (PAD), and AS3500 autosampler was used. Samples were submitted as extracts and filtered through 0.2 μm spin filters and then quantitatively transferred to 1.8 mL glass vials and diluted with water to a concentration that allows quantification from a standard curve. Samples were kept refrigerated at 4° C. Ten-microliter injections were introduced to a Dionex CarboPac PA1 guard (4×50 mm) and analytical column (4×250 mm). An auxiliary pump delivered 300 mM sodium hydroxide through a T-juncture immediately post column but before the PAD at a constant flow rate of ˜0.2 mL/minute. A six-point standard curve with a range from 0.5 μg/mL to 100 μg/mL was used for quantitation. Initial eluent conditions for sugar separation consisted of 100% water at a flow rate of 1 mL/minute. Sugars eluted in the order of arabinose, galactose, glucose, xylose, and mannose at approximately 9, 10.5, 13, 16, and 18 minutes, respectively. At 20 minutes, a step gradient consisting of 30%-water, 50%-600 mM NaOH, and 20%-300 mM NaOH/500 mM NaOAC, was used to rid the column of contaminants. At 32 minutes, a step gradient was used to return to 100% water conditions and re-equilibrate the column to initial conditions. Total run time was 43 minutes.

The cumulative results demonstrate that interference with RGP1 using a suppression cassette expressing hpRNA targeting expression of RGP1 decreases arabinose concentration in the seed.

Results

The T1 seeds were produced from T0 plants transformed with the vector described above pollinated with HC69. T1 kernels of a given ear should segregated to W:T=1:1 if there is only one insert. A total of 115 events produced T1 seeds. All of these events were screened by Western blotting in the endosperm, and only 20 events were screened in endosperm and embryo to confirm that cosuppression occurred in both tissues simultaneously. 8 kernels were screened per event, and only those events containing at least 3 WT and 3 T kernels were further processed for hemicellulose analysis.

Results from T1 Seeds.

The data from T1 generation primarily demonstrates the biological functions of RGP and the ability to manipulate its expression, which resulted in direct impact on structural changes and accumulation of hemicellulose.

The results demonstrate a reduction of hemicellulosic arabinose in endosperm and embryo. Hemicellulose analysis of a total of 73 T1 events was performed in endosperm. Arabinose was reduced in 72 events out of 73, up to a 75% reduction. In the embryo, arabinose was reduced in 50 T1 events out of 51 Ti events, up to an 80% reduction. This reduction of arabinose in hemicellulose was also reflected in the lower Ara/Xyl ratio indicating that cosuppression of RGP also results in changes in the Ara/Xyl ratio. In the endosperm, the Ara/Xyl ratio for transgenics was 0.5 in comparison to approximately 1.3 for WT. In the embryo, the Ara/Xyl ratio for transgenics was 0.3-0.8 in comparison to approximately 1.4 for WT.

As a consequence of arabinose reduction, overall hemicellulose levels were lower in a majority of T1 events, up to 50% in both endosperm and embryo.

Results from T2 Seeds.

T1 information for eleven events produced enough T2 seeds for analysis. Results mirrored those observed for T1 seeds. Thus, for both endosperm and embryo, arabinose levels, ratio of arabinose to xylose, and hemicellulose levels were reduced in transgenics relative to controls.

Experimental design of PHP20025 for T2 seeds production.

| | | | CROSSES to Produce T2 Seeds | | | | | |
|---|---|---|---|---|---|---|---|---|
| | | | Self | HC69-B | CS27 | EDE70 | EE4RD | |
| T1SID | Genotype | Parents | Male | Male | Male | Male | Male | Feature |
| 5035716 | WT | Female | 1200/6 Ears | 1200/6 Ears | 1200/6 Ears | 1200/6 Ears | 1200/6 Ears | Control |
| | T | Female | 1200/6 Ears | 1200/6 Ears | 1200/6 Ears | 1200/6 Ears | 1200/6 Ears | Transgenic |
| 5034829 | WT | Female | 1200/6 Ears | 1200/6 Ears | 1200/6 Ears | 1200/6 Ears | 1200/6 Ears | Control |
| | T | Female | 1200/6 Ears | 1200/6 Ears | 1200/6 Ears | 1200/6 Ears | 1200/6 Ears | Transgenic |
| 5035695 | WT | Female | 1200/6 Ears | 1200/6 Ears | 1200/6 Ears | 1200/6 Ears | 1200/6 Ears | Control |
| | T | Female | 1200/6 Ears | 1200/6 Ears | 1200/6 Ears | 1200/6 Ears | 1200/6 Ears | Transgenic |
| 5035694 | WT | Female | 1200/6 Ears | 1200/6 Ears | 1200/6 Ears | 1200/6 Ears | 1200/6 Ears | Control |
| | T | Female | 1200/6 Ears | 1200/6 Ears | 1200/6 Ears | 1200/6 Ears | 1200/6 Ears | Transgenic |
| 5036070 | WT | Female | 1200/6 Ears | 1200/6 Ears | 1200/6 Ears | 1200/6 Ears | 1200/6 Ears | Control |
| | T | Female | 1200/6 Ears | 1200/6 Ears | 1200/6 Ears | 1200/6 Ears | 1200/6 Ears | Transgenic |
| 5035698 | WT | Female | 1200/6 Ears | 1200/6 Ears | 1200/6 Ears | 1200/6 Ears | 1200/6 Ears | Control |
| | T | Female | 1200/6 Ears | 1200/6 Ears | 1200/6 Ears | 1200/6 Ears | 1200/6 Ears | Transgenic |
| 5035690 | WT | Female | 1200/6 Ears | 1200/6 Ears | 1200/6 Ears | 1200/6 Ears | 1200/6 Ears | Control |
| | T | Female | 1200/6 Ears | 1200/6 Ears | 1200/6 Ears | 1200/6 Ears | 1200/6 Ears | Transgenic |
| 5035691 | WT | Female | 1200/6 Ears | 1200/6 Ears | 1200/6 Ears | 1200/6 Ears | 1200/6 Ears | Control |
| | T | Female | 1200/6 Ears | 1200/6 Ears | 1200/6 Ears | 1200/6 Ears | 1200/6 Ears | Transgenic |
| 5035692 | WT | Female | 1200/6 Ears | 1200/6 Ears | 1200/6 Ears | 1200/6 Ears | 1200/6 Ears | Control |
| | T | Female | 1200/6 Ears | 1200/6 Ears | 1200/6 Ears | 1200/6 Ears | 1200/6 Ears | Transgenic |
| 5034842 | WT | Female | 1200/6 Ears | 1200/6 Ears | 1200/6 Ears | 1200/6 Ears | 1200/6 Ears | Control |
| | T | Female | 1200/6 Ears | 1200/6 Ears | 1200/6 Ears | 1200/6 Ears | 1200/6 Ears | Transgenic |
| 5035714 | WT | Female | 1200/6 Ears | 1200/6 Ears | 1200/6 Ears | 1200/6 Ears | 1200/6 Ears | Control |
| | T | Female | 1200/6 Ears | 1200/6 Ears | 1200/6 Ears | 1200/6 Ears | 1200/6 Ears | Transgenic |
| | Transgenic | Endo & | 75% | 50% | 50% | 50% | 50% | |
| | content | Emb | | | | | | |
| | Transgenic | Pericarp | 100% | 100% | 100% | 100% | 100% | |
| | content | | | | | | | |

T1 seeds are segregating 1:1=WT:T and the transgenic seeds are heterozygous.

Fiber Anylsis for the Selfed T2 Seeds.

Hemicellulose and cellulose analysis were performed in both endosperm and embryo for the eleven events. Assuming a single insert in T1 seeds, 75% of T2 seeds from transgenic ears should be transgenic. To expedite analysis, the T2 selfed seeds were not screened with Western or PCR. The analysis was conducted as described below. Importantly, similar changes in hemicellulose were observed in the T2 generation.
a. Five best ears for both the transgenic and control rows were used to form the pools.
b. Six kernels were taken from each ear.
c. 30 kernel-pools were soaked overnight in water in the refrigerator.
d. The kernels were then dissected into embryo and endosperm and the pericarp was discarded.
e. The endosperm and embryo tissues were dried overnight in the lyophilizer and ground into a fine powder.
f. Fifty milligrams of endosperm or embryo tissue was weighed in duplicate for hemicellulose analysis.
g. The pellets after hemicellulose assays were used for cellulose determination by both Anthrone and HPLC methods.
h. The transgenic pools were much easier to dissect: the embryo was easier to remove and the transgenic pool took less time to dissect.
   General Rules for Pooling T2 Seeds.
1. For a given T1 event, 6 best ears are selected from both wild type and transgenic rows
2. From 6 best ears, 200 normal kernels per ear are used to form the pool.
3. Each pool contains 1200 kernels.
4. Due to availability of number of ears and kernels, above rules are not always strictly followed.
5. As a reminder, all the T1 seeds (GS3/HC69/HC69) are produced from T0 plants (GS3/HC69) backcrossed by HC69
   Pooled Kernel Weight and Grain Compositions Measured by NIR.

The data, given as percent of control, are the average of wild-type and transgenic of all events crossed into the same genetic background. Each genetic background contains 13,000 kernels from 60 ears for both wild type pool and transgenic pool. Comparing transgenic to wild type, kernel weight was up 3.9%, oil up 4% (confirmed by NMR), and protein up 3.8%; total fermentable starch was 0.7% lower.

Wet Milling Test for Five Selfed T2 Events.

The goal of this study was to evaluate the utilities of grains with altered hemicellulose content/composition for wet milling. It is not until the present study that knowledge has been gained on how changes in hemicellulose impact other major storage materials in the kernel, such as starch, protein, and oil and how the changes would influence grain fimctionalities used for wet milling, dry grind ethanol, and digestibility for feed.

Five selfed events were selected for the test. The experiments were designed in such a way that each T1 event would produce a pair of T2 grains, ears from wild-type plants as control against ears from transgenic plants. Although all the wild-type and transgenic plants come from the same T1 event, each individual plant has its own unique genotype on top of the difference of transgene. To minimize the genetic variations in T2 grains, grains were pooled from 200 kernels/ear from 6 best ears within a row. The tests were performed with the pooled T2 grains, which only ¾ of kernels are transgenic at best for the selfed T2 transgenic pools.

The course fiber fraction from the wet milling process is primarily pericarp. On average, there was a 6.37% reduction of coarse fiber from the events indicating that pericarp fiber has been reduced in transgenic grains.

The gluten fraction from wet milling is primarily proteins from the endosperm. On average, there was a 10.6% increase of gluten yield indicating that the protein content in the endosperm of transgenic plants was increased.

The percent of kernel oil as measured by NIR was 4% higher than that of control wild-type plants. The NIR oil data was confirmed by NMR measurement.

Kernel weight was 3.9% higher in transgenic kernels than control wild-type plants.

Overall Conclusions

The results clearly demonstrate, for the first time, that RGP is involved hemicellulose biosynthesis, preferentially affecting the hemicellulosic arabinose residue. Generally, overall hemicellulose was reduced by RGP cosuppression. Further, the physical properties of the cell wall must be profoundly altered due to the reduction of arabinose side-chains and cross-linking sites. Other observations include: starch content was not affected by cosuppression of RGP; extractable starch levels remained unchanged in the transgenic grains; protein content was higher in transgenic kernels, up 3.8%; oil content was higher in transgenic kernels, up 4%; and kernel weight was increased, up 3.9%. Other results indicate that cosuppression of RGP resulted in altered hemicellulose composition and lower hemicellulose content in seeds, including embryo, endosperm, aleurone layer, and pericarp. Germ separation was much easier and cleaner for transgenic grains than their wild-type counterparts. Germ size was reduced in the transgenic grains. Endosperm size was bigger in transgenic grains. Percent of pericarp was reduced in transgenic grains.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A plant that is genetically modified to reduce the expression of reversibly glycosylated polypeptide (RGP) resulting in a reduction in arabinose levels within said plant by at least about 40% as compared to a comparable wild-type plant.

2. The plant of claim 1, wherein arabinose levels within said plant are reduced by up to about 70% as compared to a comparable wild-type plant.

3. The plant of claim 1, wherein said plant comprises at least one nucleotide construct that is capable of expressing a polynucleotide that inhibits the expression of RGP.

4. Transgenic seed of the plant of claim 1.

5. Transgenic seed of the plant of claim 2.

6. A method for altering hemicellulose composition in a plant, said method comprising introducing into said plant at least one nucleotide construct that is capable of expressing a polynucleotide that reduces the expression of reversibly glycosylated polypeptide (RGP) resulting in a reduction in arabinose levels within said plant by at least about 40% as compared to a comparable wild-type plant.

7. The method of claim 6, wherein arabinose levels within said plant are reduced by up to about 70% as compared to a comparable wild-type plant.

8. The method of claim 6, wherein said polynucleotide is stably integrated into the genome of the plant.

9. The method of claim 6, comprising:
(a) transforming a plant cell with said nucleotide construct; and
(b) regenerating a transformed plant from the transformed plant cell of step (a).

10. The method of claim 6, wherein the nucleotide construct capable of expressing a polynucleotide that reduces the expression of RGP in the plant comprises:
(a) a sense sequence consisting of at least 19 nucleotides corresponding to an mRNA encoding an RGP;
(b) a complementary nucleotide sequence having at least 94% identity to the complement of the sense sequence of (a); and
(c) a promoter that is functional in said plant.

11. The method of claim 10, wherein said promoter is a seed-preferred promoter.

12. The method of claim 6, wherein hemicellulose content is reduced in a part of said plant selected from the group consisting of seed and any part thereof.

13. The method of claim 6, further comprising the step of collecting seed from said plant.

14. The method of claim 6, wherein said plant is a monocot.

15. The method of claim 6, wherein said plant is a dicot.

16. The method of claim 6, wherein said plant is selected from the group consisting of corn, soybean, sunflower, sorghum, canola, wheat, alfalfa, cotton, rice, barley, millet, Arabidopsis thaliana, tomato, Brassica vegetables, peppers, potatoes, apples, spinach, or lettuce.

17. A plant produced according to the method of claim 6.

18. Seed of the plant of claim 17, wherein said seed comprises said nucleotide construct stably integrated into its genome.

19. The method of claim 6, wherein said plant is maize.

20. The method of claim 19, wherein said nucleotide construct comprises the suppression cassette set forth in SEQ ID NO: 1.
